SEQUENCE LISTING (5 sequences)

SEQ ID NO: 1 - DNA, 12 bp, Artificial Sequence
  Synthetically generated primer
ccannnnnntgg

SEQ ID NO: 2 - DNA, 33 bp, Artificial Sequence
  Synthetically generated primer
ggggggagggttttcccagtcacgacttaggcs

SEQ ID NO: 3 - DNA, 33 bp, Artificial Sequence
  Synthetically generated primer
ggggggagggttttcccagtcacgacttaggcn

SEQ ID NO: 4 - DNA, 33 bp, Artificial Sequence
  Synthetically generated primer
ggggggagggttttcccagtcacgacttagncc

SEQ ID NO: 5 - DNA, 23 bp, Artificial Sequence
  Synthetically generated primer
taagtcgtgactgggaaaaccct

(19) United States
(12) Patent Application Publication
(10) Pub. No.: US 2002/0102579 A1
(43) Pub. Date: Aug. 1, 2002

(54) Method for analyzing genomic DNA

(75) Inventors: Shigeo Yoshida, Saitama (JP); Tomoki Matsuyama, Saitama (JP); Tomoko Abe, Saitama (JP)

Correspondence Address: CHRIS T. MIZUMOTO, Fish & Richardson P.C., Suite 2800, 45 Rockefeller Plaza, New York, NY, 10111, US

(73) Assignee: Riken, Japan corporation

(21) Appl. No.: 09/931,323

(22) Filed: Aug. 16, 2001

Related U.S. Application Data
(62) Division of application No. 09/500,719, filed on Feb. 9, 2000.

(30) Foreign Application Priority Data
Feb. 9, 1999 (JP) 031724/1999

(51) Int. Cl. C12Q 1/68; C12P 19/34

(52) U.S. Cl. 435/6; 435/91.2

(57) ABSTRACT

A method for analyzing genomic DNA, comprising the steps of: (a) treating genomic DNA with a first restriction enzyme; (b) linking one end of an adapter to the restriction enzyme cleavage site, and labeling the other end of the adapter; (c) treating the resulting DNA fragments with a second restriction enzyme to bring about first-dimensional fractionation; (d) treating the fractionated DNA fragments of step (c) with a third restriction enzyme to bring about second-dimensional fractionation; and (e) detecting the spots of the labeled DNA fragments fractionated in step (d).

### BACKGROUND OF THE INVENTION

1. Field of the Invention

The present invention relates to a method for analyzing genomic DNA.

2. Description of the Related Art

The need for increasingly rapid genome analysis of higher animals recently has resulted in rapid progress in the development of genomic scanning techniques. Genomic scanning involves rapidly analyzing loci (gene loci) or their copy numbers, and examining the physical state of genomic DNA throughout the entire genome. Genomic scanning allows signals to be rapidly searched to determine the presence or absence of a segment on a chromosome occupied by a DNA region coding for a gene product, or a gene locus on a genome gene map. In genomic scanning, it is extremely important to focus on which markers to set up on the genome, which signals to look for in detecting given markers on the genome, and how to search for and analyze more markers. These markers are referred to as landmarks. The analysis of the positional information of landmarks on chromosomes is a major basic technique for producing physical maps or linkage maps of the human genome. Accordingly, comparative genomic scanning of two or more types of genomic DNA makes it possible to detect changes in genomic DNA such as deletions, amplifications, and translocations.

Southern blotting, PCR, and the like have conventionally been used in genomic scanning. In Southern blotting, DNA clones (genome clones and CDNA clones) are used as landmarks (DNA markers), and can be detected by signals referred to as bands. In PCR, genome loci can be detected by utilizing the amplification of certain regions ranging in size from scores of several tens of bp to several kb flanked by primers having a specific sequence.

The two methods above, however, allow only a single locus to be detected at a time. It is therefore necessary to enable several loci to be detected at a time in order to speed up and simplify genome analysis.

### SUMMARY OF THE INVENTION

An object of the present invention is to provide a method for the analysis of genomic DNA.

As a result of extensive research to remedy the above drawbacks, the inventors perfected the present invention upon discovering that genomic DNA can be analyzed by using restriction enzyme recognition sites distributed on genomic DNA as landmarks, and by adding adapters to the restriction enzyme cleavage sites, followed by first-dimensional and second-dimensional fractionation.

Specifically the present invention is a method for analyzing genomic DNA, comprising the steps of: (a) treating genomic DNA with a first restriction enzyme; (b) linking one end of an adapter to the restriction enzyme cleavage site, and labeling the other end of the adapter; (c) treating the resulting DNA fragments with a second restriction enzyme to bring about first-dimensional fractionation; (d) treating the fractionated DNA fragments of step (c) with a third restriction enzyme to bring about second- dimensional fractionation; and (e) detecting the spots of the labeled DNA fragments fractionated in step (d).

Preferred examples of the first restriction enzyme include those which are capable of cutting genomic DNA so that the 3′ end of the recognition site has a protruding sticky end. Such restriction enzymes have been reported, including, for example, BstXI, Bg1I, and MwoI.

The present invention furthermore comprises genomic DNA analytical patterns obtained by means of the methods of analysis described above. Examples of such patterns are represented in FIGS. 4, 5, 6, and 8.

This specification includes part or all of the contents as disclosed in the specification and/or drawings of Japanese Patent Application No. 99-31724, which is a priority document of the present application.

The present invention is described in further detail below.

### BRIEF DESCRIPTION OF THE DRAWINGS FIG. 1(a) to (f) are an outline of the method of analysis in the present invention;

FIG. 2(a) to (c) are an outline of the method of analysis in the present invention;

FIG. 3 is an outline of the method of analysis in the present invention;

FIG. 4 is a photograph showing the analytic results obtained by the method of analysis in the present invention for arabidopsis genomic DNA;

FIG. 5 is a photograph showing the analytic-results obtained by the method of analysis in the present invention for arabidopsis genomic DNA;

FIG. 6 is a photograph showing the analytic results obtained by the method of analysis in the present invention for arabidopsis genomic DNA;

FIG. 7 is a photograph showing the analytic results obtained by a conventional method; and

FIG. 8 is a photograph showing the analytic results obtained by the method of analysis in the present invention for rice genomic DNA;

### DESCRIPTION OF THE PREFERRED EMBODIMENTS

A basic concept in the method for analyzing DNA in the present invention is the use of restriction enzyme recognition sites as markers (landmarks) and the detection of these landmarks in the form of signals (Restriction Landmark Genomic Scanning (RLGS): I. Hatada et al., Proc. Natl. Acad. Sci. USA, 88, 9523-9527 (1991)). An adapter is added to the aforementioned restriction enzyme cleavage site, and the adapter is labeled so as to produce a signal, followed by treatment with a restriction enzyme different from the restriction enzyme used to cleave the landmark as well as by first-dimensional and second-dimensional fractionation, thereby allowing landmarks to be searched rapidly, easily, and with high precision or sensitivity.

Genome analysis generally requires efficient searches of many types of DNA. The aforementioned RLGS allows about 3000 gene loci to be searched at a time in,humans and mice, giving it an extremely high capacity to search multiple types of DNA. Although attempts to use it with plant genomes have resulted in about the same detection capacity with rice, barley, etc. as that for humans and mice, the detectability is lower with tobacco and arabidopsis. Since it is able to detect no more than 200 gene loci for arabidopsis, the number of spots is extremely low, resulting in an extremely low amount of information. Adding an adapter to the restriction enzyme site serving as the landmark in the present invention has improved the aforementioned low detectability.

The present invention comprises the following steps.

(1) Treatment of Genomic DNA With First Restriction Enzyme The method of analysis in the present invention is applicable to almost any type of genomic DNA, whether plant-derived derived or from other species (such as animals, bacteria, or yeast). Examples of genomic DNA from plants include that from rice, tobacco, arabidopsis, citrus, barley, and asparagus, although that from rice and arabidopsis are preferred.

Examples of genomic DNA from animals include that from humans, mice, and nematodes.

Examples of genomic DNA from bacteria include that from Bacillus subtilus and E. coli.

Genomic DNA can be prepared by any commonly known method (Dellaporta et al., Plant Mol. Biol. Rep. 1:19-21 (1983)).

Extracted genomic DNA is cleaved with the first restriction enzyme (this is restriction enzyme A, which is an enzyme cleaving site A in FIG. 1(a)). Examples of restriction enzymes A include those giving a mean fragment length of more than 100 kb when cleaved, specifically, those referred to as “rare cutter” restriction enzymes recognizing 6 to 8 bases, which are enzymes that recognize restriction enzyme sites occurring only in intervals of more than an average of 100 kb.

Although either a restriction enzyme which produces a sticky end with the 5′ end of the restriction enzyme site protruding when cleaved (5′ protruding type) or one which produces a sticky end with the 3′ end of the restriction enzyme site protruding when cleaved (3′ protruding type) can be used as the aforementioned restriction enzyme A, the 3′ protruding type is preferred. Examples also include those with a protrusion length of 1 base or more. Enzymes which produce fragments with a protrusion length of 2 bases or more are preferred in order to obtain sufficient signal intensity. Examples of such restriction enzymes A include those of the 5′ protruding type, such as NotI, BssHII, and AccIII, and the 3′ protruding type, such as BstXI, Bg1I, and MwoI.

In the present invention, the preferred restriction enzyme is BstXI, which is a 3′ protruding type and has N in its recognition sequence (where N represents A, G, C, or T). When a 3′ protruding type enzyme such as BstxI is used, it is not targeted in the sequenase (T7DNA polymerase) reaction during the labeling described below because a fragment other than the target DNA fragment is labeled. There are many such 3′ protruding type enzymes with a protrusion of 2 or more bases in addition to BstXI, Bg1I, and MwoI (such as AlwNI, BanII, BsiEI, BsiHKAI, Bs1I, BsmI, Bsp1286I, BsrDI, DraIII, DrdI, Pf1MI, SfiI, TSpRI, BpmI, BseRI, BsgI, and Eco57I), any of which can be used in the method of the present invention.

(2) Linking Adapter to Restriction Enzyme Cleavage Site

Conventional RLGS entails directly labeling the site cleaved by the restriction enzyme A. In direct labeling methods, however, the detectability for tobacco and arabidopsis is particularly low, as noted above, making such a method difficult to use for all of the genomic DNA. An adapter is added in the present invention to improve the detectability.

The adapter used in the present invention is designed with a sequence (ligating sequence) allowing one end to be ligated to the cleavage site of the aforementioned restriction enzyme A, while the other end is designed with a sequence (labeling sequence) allowing the adapter to be labeled. The ligating sequence and labeling sequence are single-stranded. The interval between the ligating sequence and labeling sequence is double stranded, comprising 5 to 45 bases. One consisting of 20 to 35 bases is preferred in consideration of ease of cloning. The ligating sequence can be designed corresponding to the labeling sequence of the aforementioned restriction enzyme A. The labeling sequence and the sequence of the double-stranded region can be designed as desired.

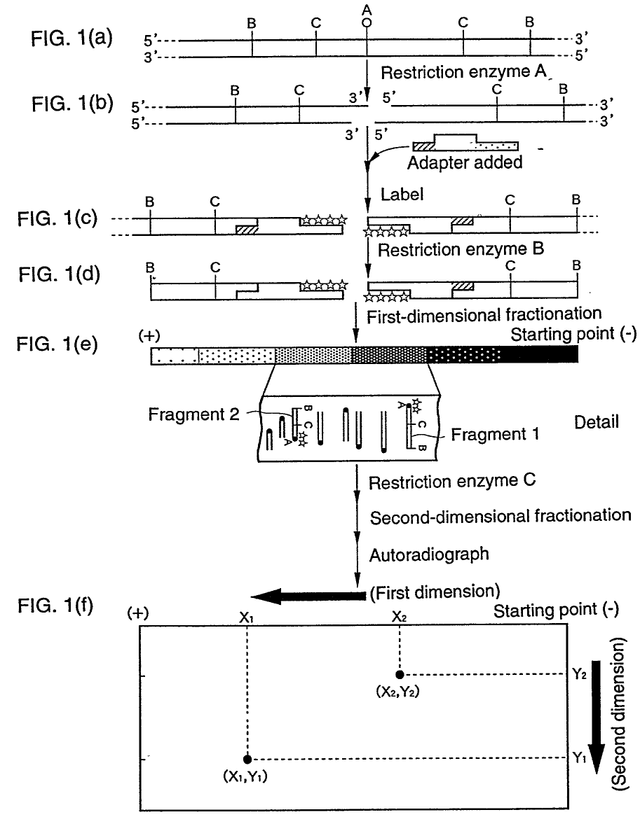

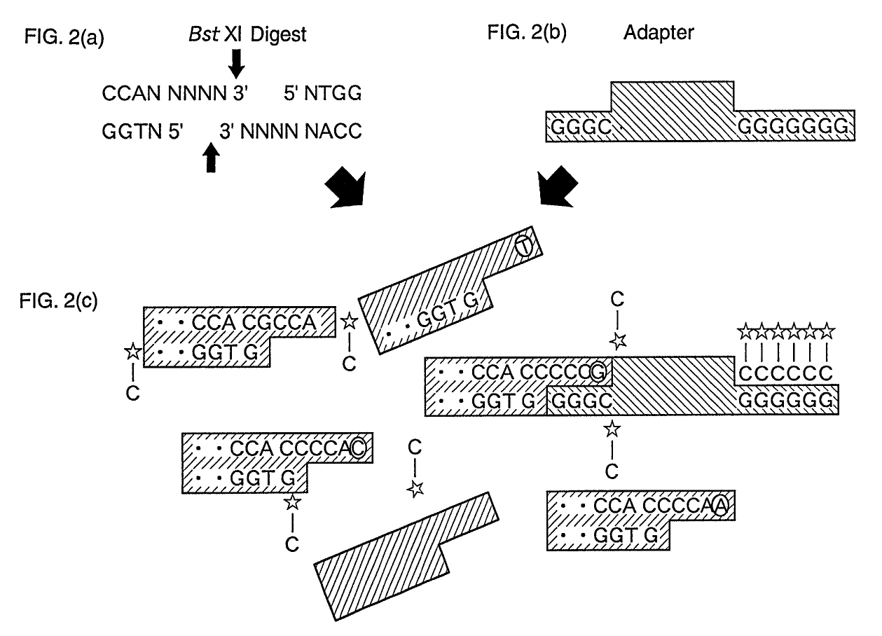

When BstXI is used as the restriction enzyme A, for example, the BstXI recognizes the following sequence, and cuts between the 8^{th} N and 9^{th} N (position shown by “*”) from the 5′ end so that the 3′ end protrudes four bases (underlined) (FIG. 2(a)).

| | |
|---|---|
| Sense strand: | 5′-CCANNNNN* NTGG-3′ (SEQ ID NO: 1) |
| Antisense strand: | 3′-GGTN *NNNNNACC-5′ |

Since N may be any of A, G, C, or T, the sequence of these four in the protrusion of the adapter can be designed with all combinations in mind (4⁴ =256). In this case, the 4 bases of the protrusion can be stipulated so as to result in a suitable number of combinations depending on the type of genomic DNA or the purpose of analysis (specifically, to detect the mutation regions of arabidopsis mutants, and the like). For example, GGGC is given as the ligating sequence of the adapter in FIG. 2(b). In this case, the adapter is ligated to one type of fragment in which the protrusion sequence is CCCG (that is, fragments having a sequence only with a protrusion CCCG) out of the fragments cut by BstXI (FIG. 2(c)). Similarly, when the ligating sequence of the adapter is GGGS (where S represents G or C), the adapter is ligated to two types of fragments in which the protrusion sequence is CCCG or CCCC, out of the fragments cut by the BStXI. The use of BStXI as restriction enzyme A accordingly allows 1 to 256 landmark sites to be selected as desired by selecting the ligating sequence of the adapter.

The adapter can be obtained using a common chemical synthesizer (such as the DNA/RNA Synthesizer model 394 by PE Applied Biosystems). The adapter is then treated with DNA ligase and ligated to the genomic DNA.

(3) Labeling the Adapter

The adapter ligated in the manner described above is labeled by being filled in with labeled bases (labeling sequence) (FIG. 1(c) and FIG. 2(c)). The labeling sequence can be designed to a desired length, allowing higher detection sensitivity to be achieved with greater labeling sequence lengths. Too long of a labeling sequence, however, sometimes results in the formation of secondary structures of DNA (such as stem loop structures or hair pin structures). The labeling sequence is thus preferably 2 to 10 bases so as to avoid forming such secondary structures.

Examples of labels include radioisotopes such as [α-³²p]dCTP and [α-³²p]dGTP, and fluorescent dyes such as tetramethyl-rhodamine-6-dUTP and fluorescein-12-dUTP, which can be selected as desired.

(4) Second Restriction Enzyme Treatment

Since the fragments cut with restriction enzyme A have a length of 100 kb or more, they cannot be fractionated by electrophoresis or the like without further modification. The fragments cut with restriction enzyme A are therefore treated with a second restriction enzyme to make shorter fragments (FIG. 1(d)). Examples of second restriction enzymes include those giving a mean fragment length of several to several tens of kb when cleaved, specifically, those which recognizing 6 bases and are enzymes that recognize restriction enzyme sites occurring in intervals of an average of several to several tens of kb (this is restriction enzyme B, which is an enzyme cleaving site B in FIG. 1). Examples of restriction enzymes B include ECORV and DraI.

(5) First-Dimensional Fractionation

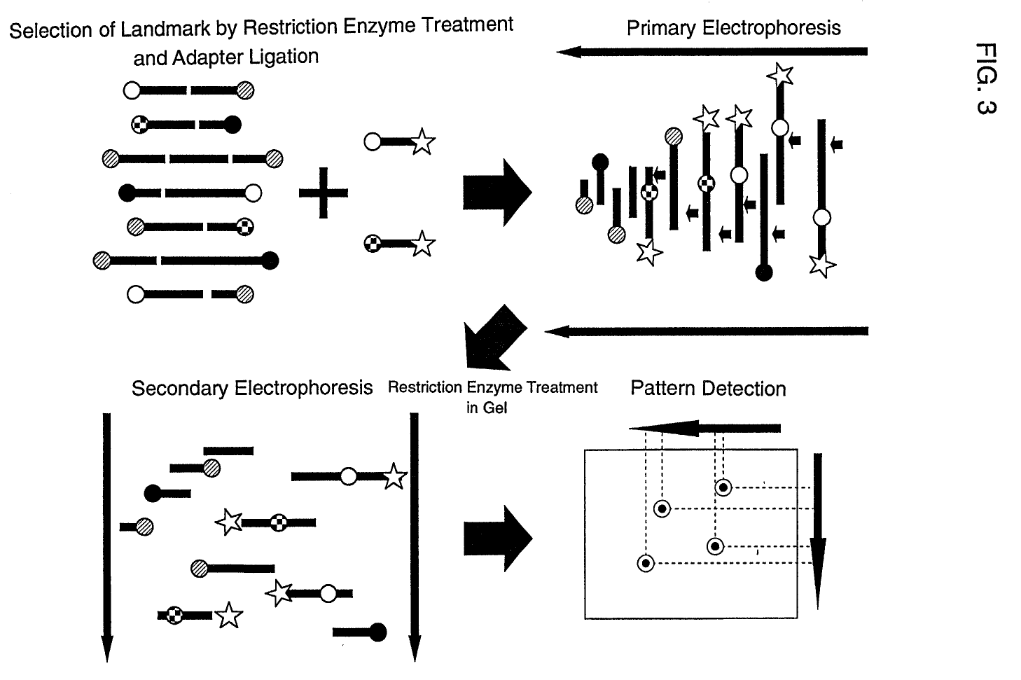

After the treatment with restriction enzyme B, the fragments undergo first-dimensional fractionation (FIG. 1(e) and FIG. 3). First-dimensional fractionation is brought about using a slender capillary tube 3 to 4.5 mm in diameter and about 60 cm in length. The fragments treated in section (4) above are injected at the point of origin of the tube in order to be fractionated. Fractionation is brought about preferably by agarose gel electrophoresis for 20 to 48 hours at room temperature (more preferably between 22 and 26° C.) in the presence of 5% sucrose.

In first-dimensional fractionation, the fragments cut by the restriction enzyme B migrate in the order of decreasing DNA fragment length from the point of origin (starting point of first-dimensional fractionation) toward the end (detail of FIG. 1(e) and FIG. 3). For example, the long fragment 1 in the detail of FIG. 1 (e) is closer to the point of origin, while the shorter fragment 2 has migrated further away from the point of origin. The position relative to the point of origin thus reflects the fragment length.

(6) Treatment with Third Restriction Enzyme

Following the conclusion of fractionation, the tube is dipped in a solution of a third restriction enzyme to treat the first-dimensional fraction products with this restriction enzyme. The third restriction enzyme has a higher cutting frequency than restriction enzymes A and B, and gives an average fragment length of several hundred bp when cut, that is, recognizes restriction enzyme sites occurring at intervals of about 300 bp (this is restriction enzyme C, which is an enzyme that cuts site C in FIG. 1

Enzymes recognizing 4 to 6 bases can be used as restriction enzyme C. Examples which can be used include MboI and HinfI.

(7) Second-Dimensional Fractionation

When the fragments are treated with restriction enzyme C, fragments flanked by restriction enzyme recognition sites A and B (referred to as A-B fragments) are cut into fragments flanked by restriction enzyme recognition sites A and C (A-C fragments) and fragments flanked by restriction enzyme recognition sites C and B (B-C fragments), resulting in DNA fragments with an average length of several hundred bp or less.

These fragments undergo second-dimensional fractionation. Examples of second-dimensional fractionation methods include methods based on polyacrylamide gel electrophoresis.

(8) Detection of Spots

Spots can be detected by a method suitable for the type of label. For example, when ³²p is used as the label, the spots can be detected by autoradiography, and when a fluorescent dye is used, the spots can be detected by a fluorescent image analyzer (such as FMBIO II Multi-view, TaKaRa). B-C fragments are not labeled after second-dimensional fractionation, and no such spots are therefore produced.

When the positions of the resulting spots are represented in terms of their distance in directions X and Y from the point of origin (X₁, Y₁), (X₂, Y₂) etc. to (Xₙ, Yₙ), the X coordinate reflects the distance from the recognition site of restriction enzyme A to the recognition site of restriction enzyme B (A-B fragments), and the Y coordinate reflects the distance from the recognition site of restriction enzyme A to the recognition site of restriction enzyme C (A-C fragments) (FIG. 1(f)). Accordingly, analysis of the pattern of spots allows the location of mutations in a genome to be specified and mutations caused by the modification of DNA to be detected. For example, since spot density reflects the number of copies of detected fragments, specific regions can be determined to be overlapping, and when position (X₂, Y₂) after second-dimensional fractionation in the pattern of spots in FIG. 1(f) has shifted or disappeared, a portion of the base sequence of the DNA fragment can be determined to have mutated (deletions, substitutions, addition, etc.).

(9) Pattern of Spots

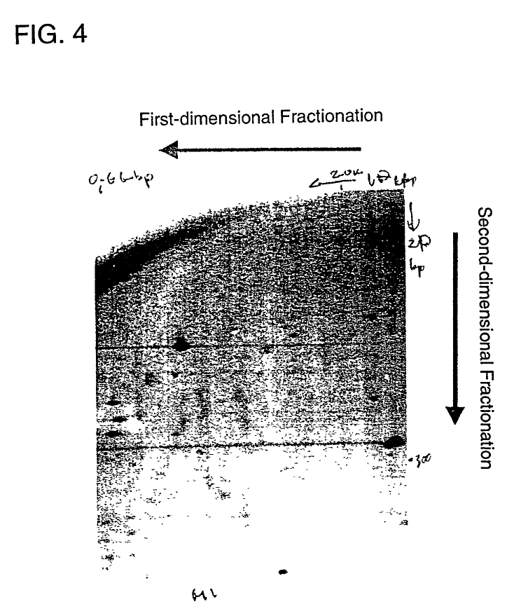

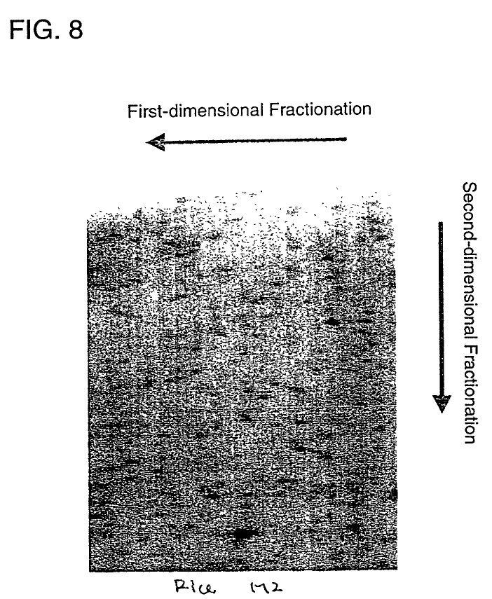

The pattern of spots obtained by the method described above is specific to the plant that has been used (characteristic of tobacco when tobacco is used, for example), and can be obtained in the form of a standard pattern for a given plant. For example, FIGS. 4 through 6 are standard patterns for arabidopsis, while FIG. 8 is a standard pattern for rice.

Furthermore, the use of a methylation insensitive restriction enzyme can provide data effective for distinguishing varieties, phyletic lines or individuals, for analyzing mutants induced by radiation or the like, or for constructing chromosomal maps. On the other hand, the C at the 5 position in so-called CG dinucleotides is often methylated in mammals, and restriction enzymes are sometimes affected by this modification and cannot cut that site (referred to as methylation insensitive enzymes). The use of such methylation sensitive enzymes (such as NotI, AccIII, and BssHII) as the first restriction enzyme allows regions of methylation-induced advanced mutations of DNA to be detected and analyzed. For example, when a cell has become cancerous or senescent, the DNA region is considered to have methylated. When a restriction enzyme such as NotI is used for this type of DNA, the methylated portion is not cut, and the resulting pattern of spots is different from the standard pattern. It is thus possible to detect genes involved in cancer, senescence, or the like.

### EXAMPLES

The present invention is described in further detail below with reference to examples, but the scope of the present invention is not limited by these examples.

### Example 1

### Genomic Analysis of Arabidopsis

In this example, the arabidopsis genome was analyzed using BstXI as the landmark restriction enzyme (restriction enzyme A).

(1) Preparation of Genomic DNA

Genomic DNA (200 mg) of Arabidopsis thaliana in aseptic culture was isolated by common SDS, and it was confirmed that there were few deletions by mini-agarose gel electrophoresis.

(2) Treatment with First Restriction Enzyme

Approximately 1 μg of the resulting genomic DNA was digested for 2 hours or more in 40 μL reaction solution using addition buffer with BstXI (Takara), followed by ethanol precipitation and DNA concentration.

(3) Design, Ligation, and Labeling of Adapter

The following mixtures were prepared in this example: a mixture of two adapters (M1 set) with GGCS sticky ends (where S is G or C); a mixture of four adapters (M2 set) with GGCN sticky ends; and a mixture of 4 adapters (M4 set) with GNCC sticky ends.

| | | |
|---|---|---|
| M1 (sense): | 5′-GGGGGGAGGGTTTTCCCAGTCACACTTAGGCS-3′ | (SEQ ID NO: 2) |
| (antisense): | 3′-TCCCAAAAGGGTCAGTGCTGAAT-5′ | (SEQ ID NO: 5) |
| M2 (sense): | 5′-GGGGGGAGGGTTTTCCCAGTCACGACTTAGGCN-3′ | (SEQ ID NO: 3) |
| (antisense): | 3′-TCCCAAAAGGGTCAGTGCTGAAT-5′ | (SEQ ID NO: 5) |
| M4 (sense): | 5′-GGGGGGAGGGTTTTCCCAGTCACGACTTAGNCC-3′ | (SEQ ID NO: 4) |
| (antisense): | 3′-TCCCAAAAGGGTCAGTGCTGAAT-5′ | (SEQ ID NO: 5) |

Phosphoric acid was added to the first base (T) of the antisense strand (SEQ ID NO: 5).

100 units of E. coli DNA ligase and 1 to 10 pmol of the aforementioned adapters were added, the reaction solution was adjusted to a final amount of 10 μL, and a ligation reaction was brought about while gently stirred for 48 hours at 16° C.

The reaction solution was then applied to a column (MicroSpin S-400 HR Column, Pharmacia Biotech) to remove the adapters which had not been ligated to genomic DNA. The removal of the adapters can be omitted. Detection is possible in the present invention with or without the removal step.

500 ng of genomic DNA with ligated adapters was treated for 30 minutes at 37° C. in the presence of [α-³²P] dCTP (60000 Ci/mmol), Sequenase Ver. 2.0 (Amersham), and buffer suitable for the reaction to label the adapters. The second restriction enzyme EcORV (Takara) was added to the reaction solution under optimal conditions to adjust the fragments to a length allowing them to be fractionated on agarose gel, and the fragments were digested for 2 hours or more at 37° C. The restriction enzyme was added in an amount of 10 units per μgram of DNA.

The resulting samples were treated for 10 minutes at 65° C. to inactivate the enzyme.

(4) First-Dimensional Fractionation

Approximately 300 ng of labeled sample was-applied on gel prepared to a final concentration of 0.8% agarose (Seakem GTG Agarose, PWC), 5% sucrose, and 1 ×1-D buffer (1 M Tris-acetate, pH 8.0, 400 mM sodium acetate, 30 mM EDTA₂Na, and 360 mM NaCl), and first-dimensional fractionation was brought about for 48 hours at 2 V/cm.

(5) Second-Dimensional Fractionation

The gel obtained upon first-dimensional fractionation was replaced with K buffer (20 mM Tris-HCl, pH 8.5, 10 mM MgCl₂, 1 mM dithiothreitol, and 100 mM KCl) (two 10 minute treatments), and it was then treated for 3 hours at 37° C. in K buffer containing the third restriction enzyme (MboI (500 unit/tube). Second-dimensional fractionation was then brought about for 22 hours at 3 V/cm using 5% acrylamide gel and 1 ×TBE (0.5 Tris-base, 0.62 M boric acid, and 10 mM EDTA₂Na). The first- and second-dimensional fractionation was managed using a genome scanning two-dimensional electrophoresis device by Biocraft.

(6) Detection of Spots

The resulting genomes were dried in a dryer (BioRad, Gel Dryer Model 583) and were then exposed for 16 hours in a BAS 2000 Image Analyzer (Fuji Film), and the patterns were checked. Favorable patterns were exposed for 7 to 21 days at −80° C. using X-ray film (Kodak, XAR5) to obtain profiles.

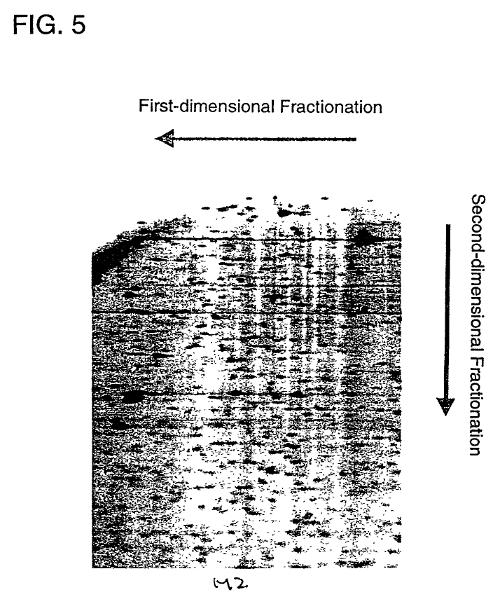

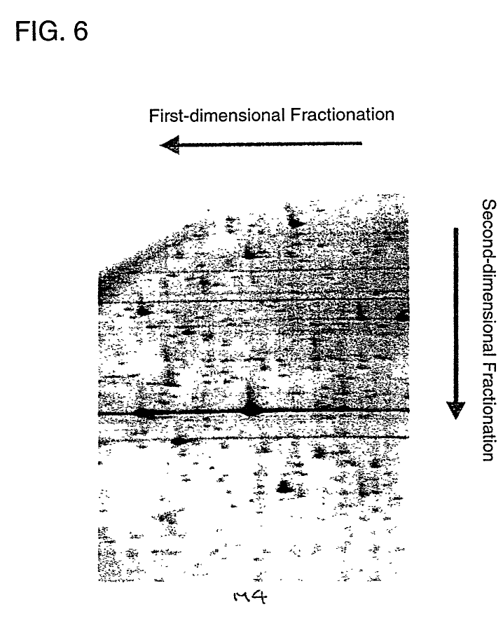

A greater number of spots was clearly found in the M2 set (FIG. 5) and M4 set (FIG. 6) than in the M1 set (FIG. 4). It may be seen that obviously different spot gene loci were detected between the M2 and M4 sets.

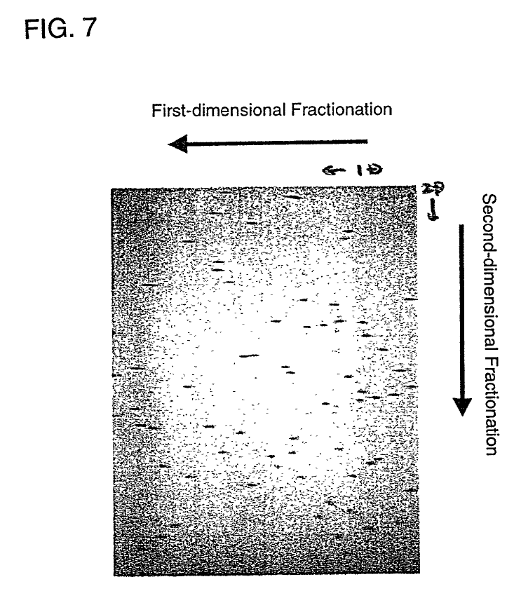

There were no more than 200 spots with the use of Not I as the landmark restriction enzyme but without using any adapter (control) (FIG. 7).

The above results indicate that an extremely large number of spots for arabidopsis was detected by the method of the present invention, (and that there were far more differing profiles of landmarks for the analysis of genomic DNA, resulting in better detection of mutations and the like of target genes.

### Example 2

### Rice Genomic Analysis

This is an example of the genomic analysis of rice using BstXI as the landmark restriction enzyme (restriction enzyme A).

Genomic DNA (200 mg) of rice (Oryza sativa L.) in aseptic culture was isolated by common SDS, and it was confirmed that there were few deletions by mini-agarose gel electrophoresis. Spots were then detected in the same manner as in Example 1 using the M2 set of adapters.

The pattern of genomic DNA for rice was obtained (FIG. 8).

The present invention provides a method for analyzing genomic DNA. The method of the present invention allows mutations and the like of target genes to be detected, allows the number of detected spots to be controlled, and can reduce the time, labor, and costs involved in screening restriction enzymes, establishing the conditions of electrophoresis, and the like.

All publications, patents and patent applications cited herein are incorporated herein by reference in their entirety.

Sequence Listing Free Text:

Sequence listing 1: n indicates a, g, c, or t (existing locations: 4) sequence listing 1: n indicates a, g, c, or t (existing locations: 5)

sequence listing 1: n indicates a, g, c, or t (existing locations: 6)

sequence listing 1: n indicates a, g, c, or t (existing locations: 7) sequence listing 1: n indicates a, g, c, or t (existing locations: 8)

sequence listing 1: n indicates a, g, c, or t (existing locations: 9)

sequence listing 3: n indicates a, g, c, or t (existing locations: 33)

sequence listing 4: n indicates a, g, c, or t (existing locations: 31)

What is claimed is:

1. A method for analyzing genomic DNA, comprising the steps of:
(a) treating genomic DNA with a first restriction enzyme;
(b) linking one end of an adapter to the restriction enzyme cleavage site, and labeling the other end of said adapter;
(c) treating-the resulting DNA fragments with a second restriction enzyme to bring about first-dimensional fractionation;
(d) treating the fractionated DNA fragments of step (c) with a third restriction enzyme to bring about second-dimensional fractionation; and
(e) detecting the spots of the labeled DNA fragments fractionated in step (d).

2. A method according to claim 1, wherein the first restriction enzyme is one which is capable of cutting the genomic DNA so that the 3′end of the recognition site has a protruding sticky end.

3. A method according to claim 1, wherein the first restriction enzyme is BstXI, Bg1I, or MwoI.

4. A genomic DNA analytical pattern, which has been obtained by means of a method of analysis according to any of claims 1 through 3.

5. A pattern according to claim 4, wherein the pattern is one represented by FIGS. 4, 5, 6, or 8.
